(19) United States
(12) Patent Application Publication
(10) Pub. No.: US 2006/0241598 A1
(43) Pub. Date: Oct. 26, 2006

(54) Center locking cross-connector with eccentric cam rod engagement

(76) Inventor: Farid Bruce Khalili, Briarcliff Manor, NY (US)

Correspondence Address: LAWRENCE CRUZ, 12 GOODHILL RD., BETHEL, CT, 06801, US

(21) Appl. No.: 11/369,122

(22) Filed: Mar. 6, 2006

Related U.S. Application Data
(60) Provisional application No. 60/659,241, filed on Mar. 7, 2005.

(51) Int. Cl. A61F 2/30

(52) U.S. Cl. 606/61

(57) ABSTRACT

A cross-connector system for connecting two rods of an implantable vertebral rod system utilizes relatively low-stress concentration contact areas between the connector elements and the rods. The cross-connector system includes a two-piece, elongated member (100) that is adjustable lengthwise and has, at each end, hook-shaped ends (103, 112) which each cooperate with a rotatable, eccentric cam plug (117, 126) having a curved surface (118, 128) that engages and secures a rod to the hook-shaped end in a manner that optimally distributes load to minimize stress concentrations.

### RELATED APPLICATIONS

This application is related to and claims priority from U.S. Provisional Patent Application 60/659,241, file on Mar. 7, 2005.

### TECHNICAL FIELD

The present invention relates to surgically implanted devices and, more particularly, implantable devices for vertebral repair or reconstruction comprising cross-connectors for rod constructs.

### BACKGROUND OF THE INVENTION

Various known techniques and systems exist for repairing or reconstructing injured or diseased vertebral sections in which one or more implants are provided in or adjacent to a vertebral disc space in a manner in which two or more adjacent vertebrae are stabilized relative to each other or are fused together. Such a solution provides stability and pain relief. It is known to use rod systems that generally comprise one or more metal rods that each span a gap between at least two adjacent vertebrae, wherein the rod is fixed to the adjacent vertebrae by bone screws or fasteners inserted directly into the vertebrae. It is further known to supplement such rod systems with what is referred to in the art as “cross-connector” devices which form a bridge between parallel rods in order to stabilize them relative to each other and improve overall torsional stiffness in a vertebral system that has a rod system installed. Known cross-connectors are often designed as simple metal bar sections that can be bent and cut to desired length, and that are attached to installed rods via a hook and some type of locking element. Typically, such rods comprise two parts that are slidingly adjustable relative to each other to facilitate installation when hooked ends need to be placed over parallel rods and then drawn closer to each other. For example, referring to FIG. 1, a prior art cross-connector (10) includes an elongated section (12) made of two relatively sliding parts and having at each end (only one end is illustrated) a hook (14) for engaging a rod (16) that is part of a conventional rod system and that is installed into a patient's™s vertebral system. A fastener element (18) comprises a screw-like member having threads (20) and a conical contact surface (22). After placement of each hook (14) over and in engagement with a rod (16), the hooks (14) are drawn toward each other by adjusting the two parts of the elongated section (12) to pull the rods (16) toward each other so that the elongated section (12) is in tension. After securing each hook (14) to the rods (16), the fastener elements (18) are moved into engagement with the rods (16) by turning them so that the threads (20) cause the contact surfaces (22) to engage the rods (16).

Prior art systems of the type illustrated in FIG. 1 have undesirable properties including high stress concentrations at the contact areas between the contact surfaces (22) and the rods (16), potentially leading to early failure of the rods or fastener elements.

### OBJECTS AND SUMMARY OF THE PRESENT INVENTION

It is desirable, therefore, to provide a system and technique of providing cross-connectors for vertebral rods that overcome the shortcomings of known systems, as described above, as well as other shortcomings. This object and other objects and advantages are inherent to the present invention described herein.

The present invention is directed to a cross-connector system for connecting two rods of an implantable vertebral rod system of a known type, wherein the cross-connector system of the present invention utilizes relatively low-stress concentration contact areas between the connector elements and the rods. In a preferred embodiment, the cross-connector system according to the present invention includes a two-piece, elongated member that is adjustable lengthwise and has, at each end, hook-shaped ends which each cooperate with a rotatable, eccentric cam plug having a curved surface that engages and secures a rod to the hook-shaped end in a manner that optimally distributes load to minimize stress concentrations.

### BRIEF DESCRIPTION OF THE DRAWINGS

FIG. 1 is a schematic, partial front view of a prior art cross-connector system for vertebral rod systems.

FIG. 2A is a schematic, partial front view of a cross-connector system according to a first embodiment of the present invention showing a cam plug in a non-engaged position.

FIG. 2B is a schematic, partial front view of a cross-connector system according to a first embodiment of the present invention showing a cam plug in an engaged position.

FIG. 3 is an exploded view of a cross-connector system according to a first embodiment of the present invention.

FIG. 4A is a top view of a cross-connector system according to a first embodiment of the present invention.

FIG. 4B is a front view of a cross-connector system according to a first embodiment of the present invention.

FIG. 4C is a bottom view of a cross-connector system according to a first embodiment of the present invention.

FIG. 4D is a cross-sectional view of a cross-connector system according to a first embodiment of the present invention.

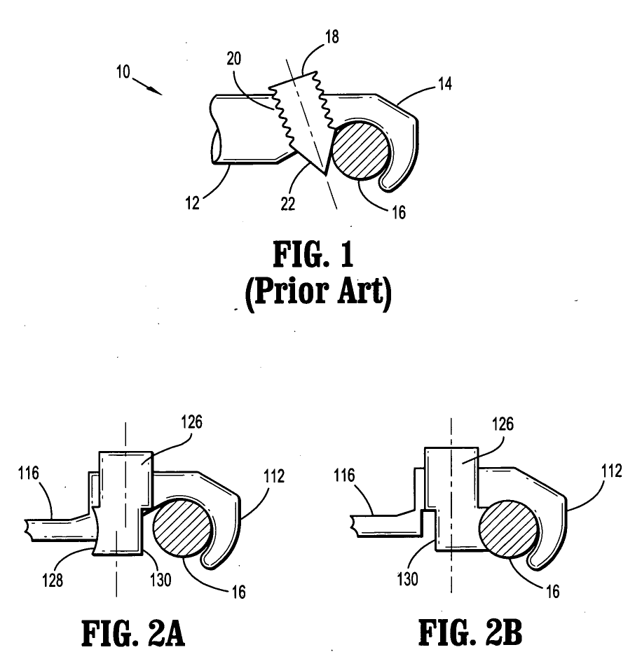

### DESCRIPTION OF THE PREFERRED EMBODIMENTS

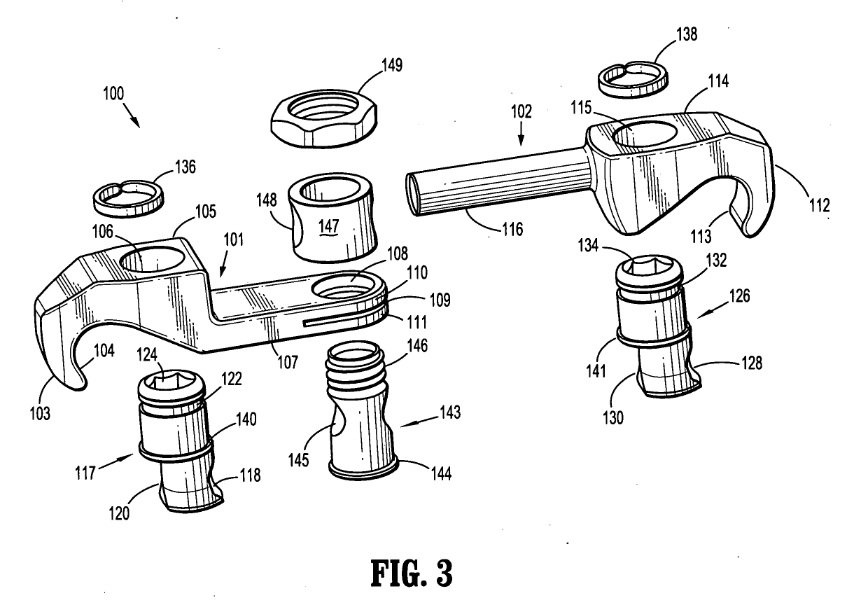

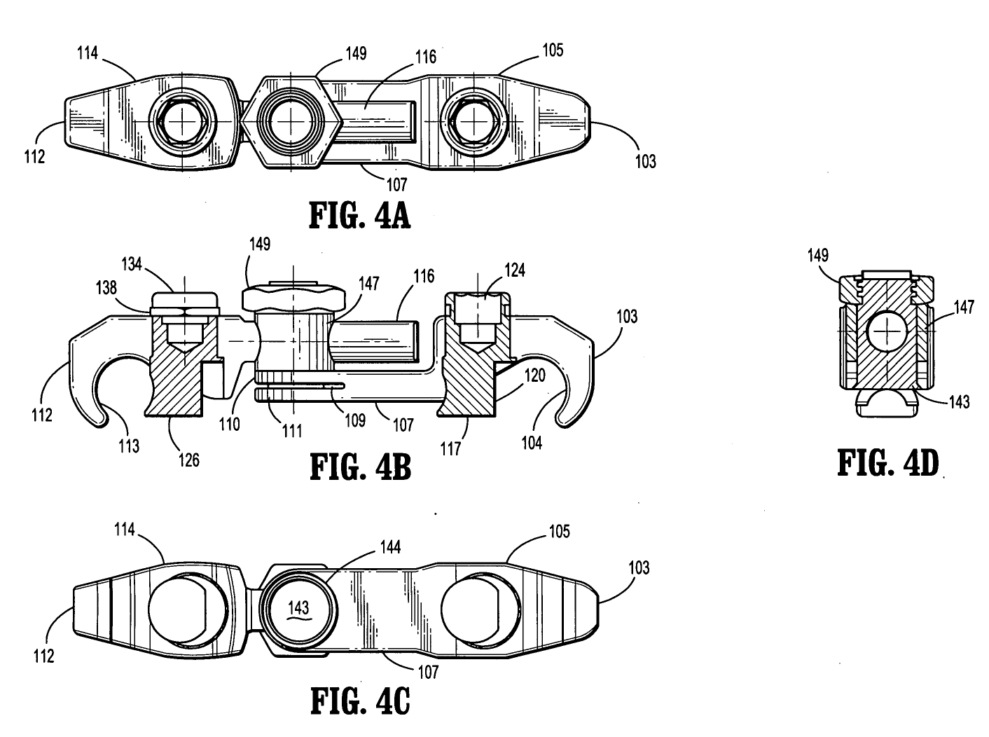

A first embodiment of the present invention is shown in FIGS. 2A-4D. According to the present invention, a cross-connector system (102) includes a first member (101) and a second member (102). The first member (101) comprises: a hook end (103) having in inner, curved rod-engagement surface (104); a cam-retaining section (105) with a hole (106) therethrough; and an arm section (107) having a hole (108) therethrough and a slot (109) forming two flexible end portions (110, 111). The second member (102) comprises: a hook end (112) having in inner, curved rod-engagement surface (113); a cam-retaining section (114) with a hole (115) therethrough; and an arm section (116) formed in the shape of a rounded rod. The system (100) further includes a first cam plug (117) having a curved, rod-engaging surface (118), an opposite non-engaging surface (120), a snap-ring channel (122), a flange (140), and a torque tool engaging section (124). Similarly, a second cam plug (126) has a curved, rod-engaging surface (128), an opposite non-engaging surface (130), a snap-ring channel (132), a flange (141), and a torque tool engaging section (134). A first snap-ring (136) locks the first plug (117) into a secured position through the hole (106) in the first member (101) cam-retaining section (105) as shown in FIG. 4B. A second snap-ring (138) locks the second plug (126) into a secured position through the hole (115) in the second member (102) cam-retaining section (114) as shown in FIG. 4B. The system (100) further includes a generally cylindrical central pivot plug (143) having a flange (144) at its lower end, a central hole (145), and threads (146) at its top end. The system (100) also includes a generally cylindrical sleeve (147) that fits around the pivot plug (143) and that has a central hole (148). A threaded nut (149) is also provided for mating with the threads (146) of the pivot plug (143).

When assembled, as shown in FIGS. 4A-4D, the pivot plug (143) is placed through the hole (108) of the arm section (107) of the first member (101). The flange (144) is sized larger than the hole (108) to prevent the plug (143) from passing all the way through the hole (108). The sleeve (147) is place over the plug (143) and its central hole (148) is aligned with the central hole (145) of the plug (143). The rod section (116) of the second member (102) is inserted through the aligned holes (145, 148) as shown in FIGS. 4A-4C. The nut (149) is threaded over the threads (146) of the pivot plug (143). The nut (149) can be tightened to retain the rod section (116) tightly so that the first member (101) and second member (102) are held in position relative to each other. The respective holes (145, 148) are sized slightly larger than the rod section (116) and, preferably, are elongated into a slot-shape so that, as the nut (149) is tightened, the sleeve (147) advances downwardly while the plug (143) is drawn further into the nut (149), causing the rod section (116) to be clamped at its top side by the hole (148) of the sleeve (147) and clamped at its bottom side by the hole (145) of the plug (143). This is facilitated by the slot (109) and flexible ends (110, 111), since the flexible ends (110, 111) will compress toward each other, closing the slot (109), as the flange (144) and the nut (149) are drawn closer to each other.

Before fully tightening the nut (149) and locking the first member (101) and second member (102) relative to each other, the hook ends (103, 112) of each member are placed over and in engagement with generally parallel rods in a vertebral rod system. The rod-engagement surfaces (104, 113) are drawn together to positively contact the rods by adjusting the relative positions of the first and second members (101, 102) with respect to each other and locking them relative to each other as described above. During this step, each cam plug (117, 126) is pivoted in a manner that their respective non-engaging surfaces (120, 130) face the rods. Then, using an appropriate torque instrument, the plugs are engaged at torque tool engaging sections (124, 134) and caused to turn so that the rod-engaging surfaces (118, 128) engage the respective rods in and interference-fitting manner. This is achieved because, as shown, the cam plugs (117, 126) function as eccentric cams. The shape of each respective rod-engaging surface (118, 128) is curved in similar contour to the rods so that surface contact and load distribution areas are maximized, thus reducing stress concentrations.

While the preferred embodiments have been herein shown and described, it is understood that various modification can be made without departing from the scope of the present invention.

What is claimed is:

1) A locking assembly for engaging and locking to a rod having a generally circular cross-section, said assembly comprising:
a hook-shaped rod-engaging component having a first curved surface for engaging a first portion of said rod;
a rotatable locking component rotatably mounted to said hook-shaped component and having a central axis about which said locking component is rotatable, and having a second curved surface for engaging a second portion of said rod wherein said second curved surface is radially spaced from said central axis at a first distance.

2) An assembly according to claim 1, further comprising
a non-engaging surface of said rotatable locking component located on a side of said locking component generally opposite of said second curved surface with respect to said central axis, and being radially spaced from said central axis at a second distance that is less than said first distance.

3) An assembly according to claim 1, further comprising
a tool-engaging surface on said rotatable locking component for selectively engaging said rotatable locking component with a tool to impart torque to cause said rotatable locking component to rotate.

4) A cross-connector locking assembly for attaching two generally parallel rods, said assembly comprising:
a first locking assembly comprising
a first hook-shaped rod-engaging component having a first curved surface for engaging a first portion of a first rod;
a first rotatable locking component rotatably mounted to said first hook-shaped component and having a first central axis about which said first locking component is rotatable, and having a second curved surface for engaging a second portion of said first rod wherein said second curved surface is radially spaced from said first central axis at a first distance;
a second locking assembly comprising
a second hook-shaped rod-engaging component having a third curved surface for engaging a first portion of a second rod;
a second rotatable locking component rotatably mounted to said second hook-shaped component and having a second central axis about which said second locking component is rotatable, and having a fourth curved surface for engaging a second portion of said second rod wherein said fourth curved surface is radially spaced from said second central axis at a second distance; and
a central locking assembly comprising
a bolt engaging an extending portion of said first hook-shaped component and engaging an extending portion of said second hook-shaped component;
a fastener engaging said bolt in a manner in which said respective extending portions are locked relative to each other.

5) An assembly according to claim 4, further comprising
a first non-engaging surface of said first rotatable locking component located on a side of said first locking component generally opposite of said second curved surface with respect to said first central axis, and being radially spaced from said first central axis at a third distance that is less than said first distance; and
a second non-engaging surface of said second rotatable locking component located on a side of said second locking component generally opposite of said fourth curved surface with respect to said second central axis, and being radially spaced from said second central axis at a fourth distance that is less than said second distance.

6) An assembly according to claim 5, further comprising
a first tool-engaging surface on said first rotatable locking component for selectively engaging said first rotatable locking component with a tool to impart torque to cause said first rotatable locking component to rotate; and
a second tool-engaging surface on said second rotatable locking component for selectively engaging said second rotatable locking component with a tool to impart torque to cause said second rotatable locking component to rotate.

7) An assembly according to claim 4, further comprising
a through-hole in said bolt;
a sleeve positioned around said bolt and having an opening on each side generally aligned with said through-hole, wherein said extending portion of said first hook-shaped component is received through said through-hole and through said openings.

8) An assembly according to claim 7, further comprising
a hole in said extending portion of said second hook-shaped component for receiving said bolt therethrough.

9) An assembly according to claim 8, further comprising
at least one slot in said extending portion of said second hook-shaped component, said slot being located near said hole enabling said extending portion of said second member to resiliently deform when said fastener is tightened relative to said bolt.
